(19) United States
(12) Patent Application Publication
(10) Pub. No.: US 2006/0047251 A1
(43) Pub. Date: Mar. 2, 2006

(54) Medical small-bore tubing system and kit

(76) Inventors: Philip Bickford Smith, Rawdon (GB); Alan Wagstaff, Bradford (GB)

Correspondence Address: SHELDON & MAK, INC, 225 SOUTH LAKE AVENUE, 9TH FLOOR, PASADENA, CA, 91101, US

(21) Appl. No.: 10/532,772

(22) PCT Filed: Oct. 22, 2003
(86) PCT No.: PCT/GB03/04566

§ 371 Date: Apr. 21, 2005

(30) Foreign Application Priority Data
Oct. 22, 2002 (GB) 0224515.7

(51) Int. Cl. A61M 5/31

(52) U.S. Cl. 604/240; 604/93.01

(57) ABSTRACT

A small bore tubing system employs affordance to assist distinction between different applications of medical tubing and their interconnections, as well as unique keying to avoid misconnection between tubing of different applications. Said affordance is by shape of a grip on connectors of the system, which shapes provide both visual and tactile application-specific affordance (eg. spine and ribs to indicate neuraxial application). Further mechanism affordance ensures appropriate connection mechanism is employed. A kit of components of a medical small-bore tubing connection system has a first converter (14), a second converter (30), a syringe (10) and a needle (50). Each converter (14, 30) has a through bore, a standard female (16) and male (36) connector, and a “different” male (18) and female (32) connector element. The first converter (14) has a latching mechanism (control ring) (20) on the different male connector (18) and which is adapted to engage a flange (34) of the corresponding female connector. Said standard connectors are 6% luer connectors. To the standard outlet of the syringe is permanently secured a first converter (14′). The hypodermic needle has said different female connector (32′) formed directly thereon. The control ring comprises a threaded (26) collar and said flange comprises thread elements (34). The control ring is axially slidable between limits (22, 24), and is rotatably free, on the first converter.

This invention is in the field of medical small-bore tubing, in particular, to connectors therefor that interconnect different tubing and component articles of the system (that is to say, items such as needles, cannulae, filters, pumps, syringes, medicine and sample collection containers etc).

A patient in intensive care in a hospital may have a multitude of various tubes and wires connected to him or her. Misconnection of any of the tubes is, at best, undesirable. But, at times, it can be serious and may even be fatal. This applies particularly to the neuraxial system of a patient. Injection of a significant number of different fluids that may be quite harmless (indeed beneficial and normal) when injected intravenously or intramuscularly or subcutaneously, are nevertheless fatal or seriously harmful when injected neuraxially. Thankfully, misconnections are relatively rare. However, when a patient has a number of tubes connected to different parts of the body, a great deal of care has to be taken to ensure that a drug or infusion intended for one region of the body is not mistakenly directed elsewhere. Doctors and nurses are trained to understand the risks and to avoid these dangers. Labels are used extensively to minimise the chance of an error, but, regrettably, mistakes are inevitable and do occur from time to time.

Mistakes are inevitable because, instead of unique connectors being used for different classes of tubular connection to the human body, there is, in fact, almost universal interchangeability. The ubiquitous 6% luer connector is employed in devices designed to access nearly every functional system within the body, for example, the gastro-intestinal, the vascular, the respiratory and the neuraxial systems.

Indeed, it is the very ubiquity and widespread application of the 6% luer connection that has rendered the introduction of different couplings so problematic.

There is enormous inertia that resists a change of this magnitude. Such inertia prevents the introduction of arrangements that could eradicate the risk associated with misconnections. The inertia is caused by several factors ranging from fear of the commercial risk of a new system, safety issues around a new arrangement, ignorance and training issues, uniform introduction, and cost. Until such time as agreed national and/or international standards dictate certain forms, and until an agreed timetable for change is implemented (in which suppliers, health authorities and medical staff are all involved) no change is likely to happen.

WO-A-9964103 discloses a system which at least minimises the risk of misconnections. It discloses first and second converters having at one end a standard male or female connector for connection to existing medical connection systems. On one converter there is a different male connector element having a key, and on the second converter there is a corresponding keyed female connector to which only the appropriate male connector can connect.

However, although this rules out actual misconnections, it does not provide a complete solution. For that, a system that assists the elimination of attempts to make a misconnection needs to be provided and it is an object of the present invention to provide such a system.

In accordance with the present invention there is provided a system of medical small bore tubing for multiple different applications, the system in each application comprising connectors between tubing of the system and/or components of the system, wherein said connectors comprise:
a male component having a stub, a first key and a through-bore for the passage of fluid to be transported; and
a female component having a stub, a second key and a through-bore for the passage of fluid to be transported;
said male and female components being adapted to be interconnected in a fluid-tight manner with inter-engagement of said first and second keys, and said stubs being adapted for connection to tubing of the system or components of the system, and at least one of said male and female components having a grip; wherein, in each application:
a) first and second keys are unique to each application of the system so that they prevent connection of a female component of one application to a male component of another application; and
b) said grip has application affordance unique to the application for which it is intended, which affordance comprises both visual and tactile cues; whereby
   not only are misconnections between tubing and components of said different applications of the system prevented but also attempts by users at said misconnection are discouraged by said affordance of said grip.

Said keys in the system may simply comprise differently shaped mating surfaces of the male and female components, so that components for one application cannot be mated in a fluid-tight manner with components for another application. Indeed, preferably, applications for the vascular system retain the existing “standard” 6% luer fitting, whereas the design of mating surfaces of connectors within the system not intended for use with the vascular system are incompatible with the standard 6% luer fitting.

Preferably, said application affordance comprises shape of the grip that is suggestive of the part of a human body for which the application is intended.

A first application of the system of the invention may be for the neuraxial system, in which event said shape of the grip is generally cylindrical having a longitudinal spine and encircling ribs suggestive of the human spine and ribs.

A second application may be for the respiratory system, whereupon said shape of the grip is generally cylindrical having alternating frusto-conical sections suggestive of a bellows.

A third application is for the enteral system, and said shape of the grip is then generally cylindrical with bulges down its length suggestive of the human colon.

Said visual and tactile cues of the application affordance are preferably provided only by said shape of the grip.

Users of the system are then guided by the affordance which not only identifies the particular application in question, but also assists user recall of that application by providing a prompt appropriate to the application. Moreover, the affordance is not merely visual, but also tactile, which has important consequences in circumstances where connections have to be made blind because they are conducted under the sheets of a hospital bed, for instance.

Said grip may also comprise mechanism affordance unique to the method of interconnection between said male and female components. Said method of interconnection may comprise a twisting step, in which case said mechanism affordance comprises a wing of said grip. On the other hand, or as well, said method of interconnection may comprise a pushing step, in which case said mechanism affordance comprises a waist of said grip. Said method of interconnection may also comprise a locking step, and then said mechanism affordance comprises a button of said grip. More than one button may be provided.

Accordingly, the present invention provides an arrangement where misconnections, at least between vitally different applications such as respiratory, enteral and neuraxial systems, can be eliminated. Further, it reduces the time spent by medical and nursing staff fruitlessly wasting time by attempting to make a connection that will not actually succeed by providing identification of the different connectors. The identification is, moreover, suggestive of the correct application. The identification is both visual and tactile. And finally, affordance can also be provided of the type of action needed to make the connection.

While in many instances, affordance-endowed grips will be provided on both male and female components of the system, this may not be practical on all components to be employed in the application.

In a preferred embodiment of the present invention, there is provided a kit of components of a medical small-bore tubing connection system as defined above, the kit comprising:
a first converter having a through bore, and a standard female connector, a different male connector element and a latching mechanism on the different male connector adapted to engage a flange of a corresponding female connector to which said different male connector is sealingly mateable; and
   a second converter having a through bore, and a standard male connector, a different female connector that corresponds with the different male connector of said first converter and a flange adapted for engagement with the latching mechanism of said first converter.

The standard connectors may be 6% luer connectors. Said different connectors may be reduced-diameter 6% conical connectors, or parallel sided connectors, which, in any event, are designed to prevent fluid tight mating with the standard 6% luer system. Preferably, one different connector may comprise a 5% taper having a 3.5 mm diameter at the tip of the male connector.

The kit may include an adapted standard 6% luer fitting syringe to the outlet of which said first converter is permanently secured. Said permanent securing may be effected by welding or adhering said first converter to such outlets, for example by ultrasonic welding.

Preferably, said syringe is simply provided with an outlet comprising said different male connector.

The kit preferably comprises needles provided with said different female connectors formed directly thereon.

The present invention also includes syringes adapted for connection to said second converter, as well as needles adapted for connection to said first converter.

In the context of the present invention, a “standard” connection is one which is commonly employed in various, non-specific medical applications. In the United Kingdom health service, that is 6% luer connectors. A “different” connection is one that will not connect to a standard connection.

Thus, the present invention provides for a low-cost introduction of a safety enhancement to a series of small-bore tubing connectors, each dedicated to a single physiological system within the body, such as the respiratory, enteral and neuraxial systems, as distinct from the vascular system which could retain the existing system, or subsequently itself be adapted with the affordance principles proposed for the other systems. It is low-cost for two fundamental reasons:

The first is that, by virtue of the provision of first converters that are adapted for permanent connection to syringes, current tooling producing syringes having normal 6% luer male outlets (or other standard outlets) can continue to operate. This provision can continue until both the tooling is worn out (thereby reducing cost and improving efficiency) and the new system has proved itself (thereby avoiding the risk of having to make changes to various syringe toolings if the different connection arrangements need modification).

The same reasoning also applies to the needles, although to a lesser extent. The tooling that makes the moulded parts of needles is no more complicated than the tooling needed for making the second converter. It may be simplest just to modify the existing tooling that makes the needle hub, and this would reduce the size of the hub.

The second reason why the present invention is a low-cost option for the introduction of the safer system is that the first and second converters provide the opportunity to employ existing components. With the converters it is possible to connect, between a syringe and needle (that are each irrevocably adapted as described above), any number of existing components currently in use and having normal 6% luer (or other standard) connections. Such components include filters, tubes, stopcocks, junctions etc.

It may be thought, however, that, if converters provide the opportunity for standard fittings to be employed, they also provide opportunities for abuse of the system. However, no system can prevent abuse, and that is not the object of the present invention. On the other hand, it is not proposed to make converters generally available except to manufacturers of components that are frequently employed in medical tubing conduits. The converters would be permanently secured to these components thereby permanently converting them into the new system. Again, without the high costs and inherent risk of adapting tooling to a new design before it is universally accepted.

The object of the present invention is firstly to introduce a system where the opportunity for mistakes to be committed is minimised and, crucially, to introduce such a system gradually so that the commercial risks and costs are minimised. This means that a system can be introduced much more quickly than if the whole system needs to be changed at the same time. It needs also to be borne in mind that the necessity for a change of design is only slowly developing. This is because errors are infrequently made. The commercial balance between addressing the causes of those errors, on the one hand, and meeting the financial consequences when they occur, on the other, is still being weighed. Only slowly is the balance moving in the favour of the former approach, and the present invention helps to shift that balance firmly in that direction.

Thus, the final solution may be, in time, unique tubing, filters, other accessories, needles, catheters, syringes etc. An additional advantage accruing from the present invention is that it opens the possibility of drug containers being adapted or converted to incorporate the dedicated connector shapes. This is important where a drug is restricted to one route of delivery for a specific physiological system. Until then, however, the present invention provides an opportunity to start the process, as well as providing a means for identification and, additionally, fast learning, of the different applications of the system.

Preferably, said latching mechanism comprises a threaded collar and said flange comprises thread elements. The collar is preferably axially slidable between limits, and rotatably free, on the first converter. The action of the threaded collar and the mating of the different connector mimic that of the standard 6% luer connectors. Thus they minimise in-service training requirements, whilst ensuring correct usage from previous experience.

Preferably, the latching mechanism comprises said grip. It is this that is visually coded to identify the class of medical applications for which it is intended.

In another aspect, the present invention provides a component of medical tubing to which a first and second connector of a kit as defined above has been connected to male and female standard connections of said component.

Preferably, said connections are rendered permanent by application of adhesive between a latching mechanism on the component and the standard female connector of the first converter and between the latching mechanism of the second converter and the female connector of the component.

Said component may be a filter, valve or tube junction.

In yet another aspect, the present invention provides a method of introducing into use a new connection system for an existing medical small bore tubing system that employs standard male and female connectors adapted to be sealingly mated together, said method comprising the steps of:
a) providing a kit as defined above;
b) permanently connecting the standard female connectors of said first converters to the standard male connectors of components of said existing system; and
c) permanently connecting the standard male connectors of said second converters to the standard female connectors of components of said existing system.

Said permanent connection is preferably by ultrasonic welding. Alternatively, where standard male connectors employ an integral latching mechanism, said permanent connection is preferably by adhesion through adhesive disposed between the inside of said latching mechanism and the outside of said standard female connector.

Embodiments of the invention are described hereinafter, by way of example, with reference to the accompanying drawings, in which:—

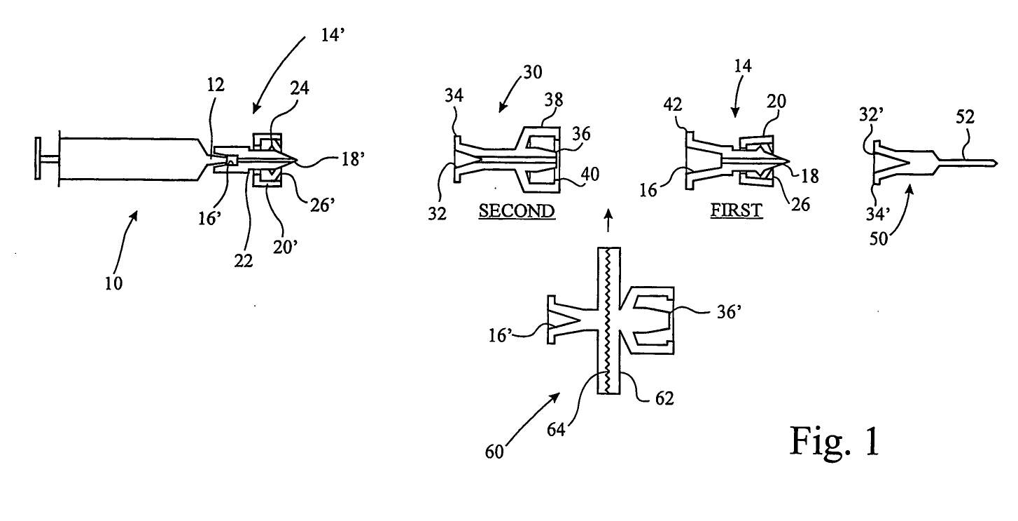

FIG. 1 is a schematic illustration of a kit in accordance with one aspect of the present invention, in use;

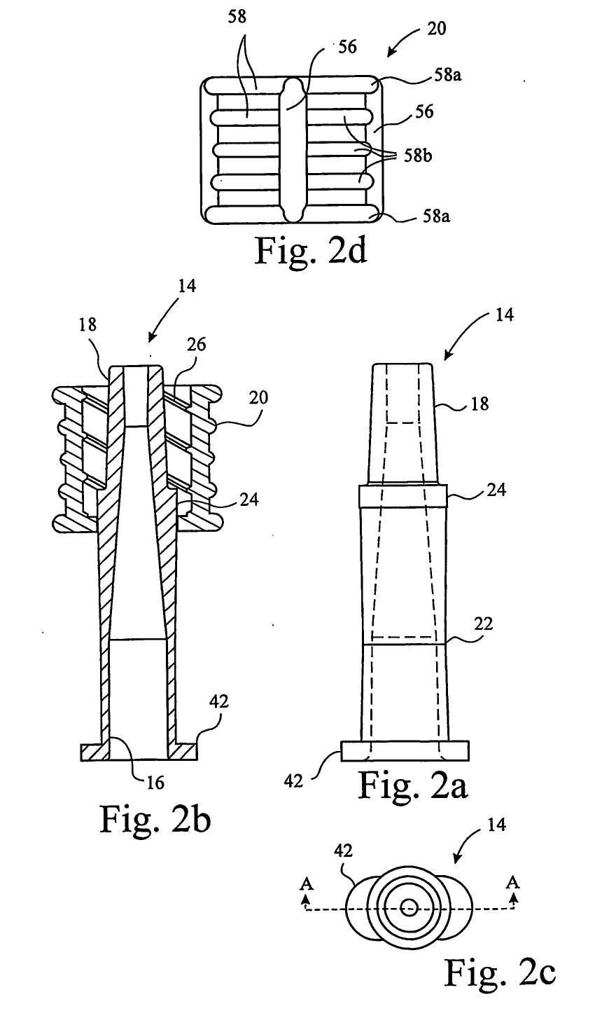

FIGS. 2a, b and c are a side view (with latching mechanism removed), a side section on the line A-A in FIG. 2c, and an end view, respectively, of a first converter in accordance with the present invention, FIG. 2d being a side view of a latching mechanism of the first converter;

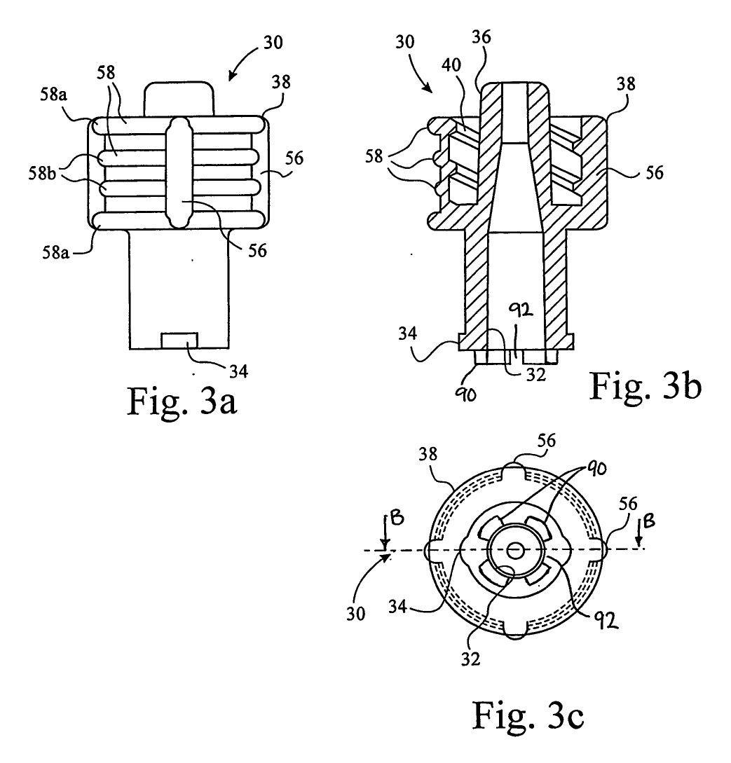

FIGS. 3a to c are a side view, a side section on the line B-B in FIG. 3c, and an end view, respectively, of a second converter in accordance with the present invention;

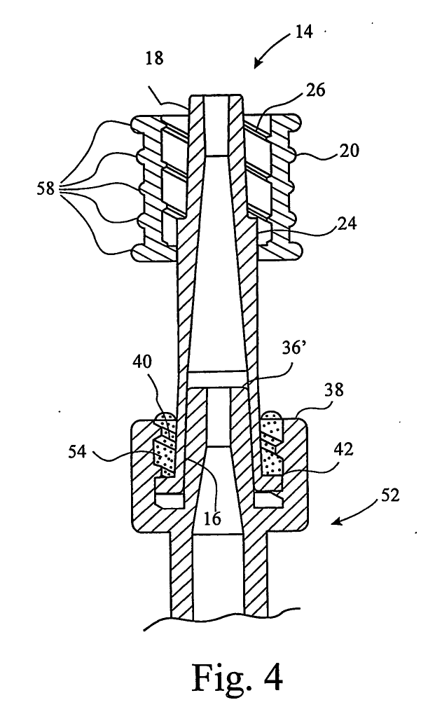

FIG. 4 is a section through a permanent connection between standard male and female connectors;

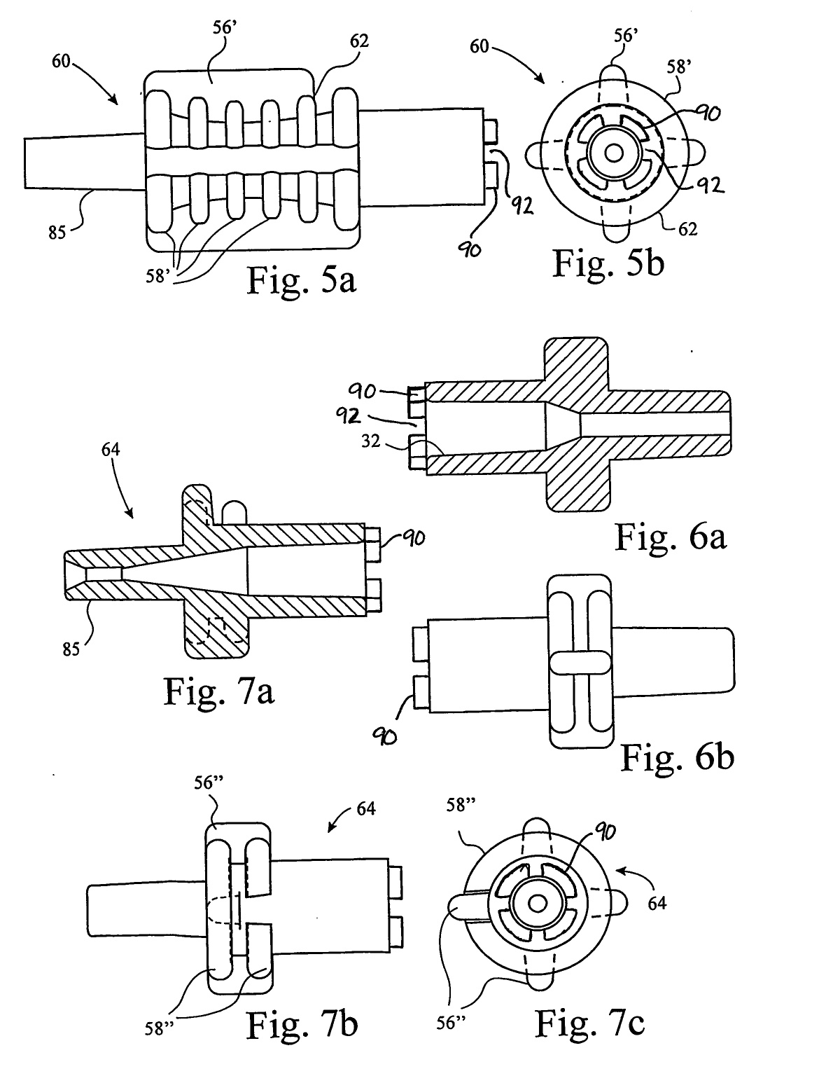

FIGS. 5a and b are a side view and an end view of SUBSTITUTE SHEET (RULE 26) an epidural needle hub in accordance with the present invention;

FIGS. 6a and b are a side section and side view of a needle adaptor;

FIGS. 7a b and c are a side section, side view and an end view of a neuraxial needle hub in accordance with the present invention;

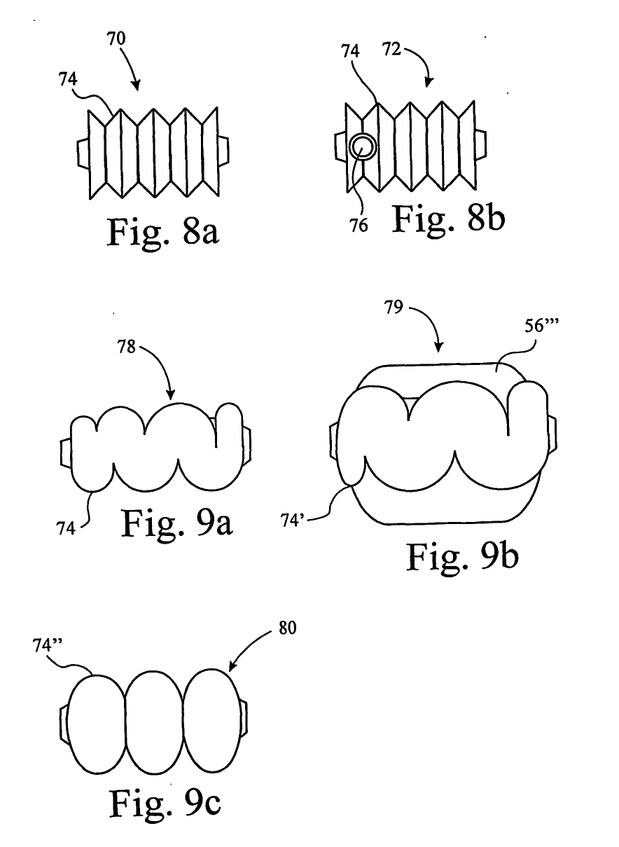

FIGS. 8a and b are schematic side-view illustrations of possible respiratory connectors, or grips therefor, in accordance with the present invention; and

FIGS. 9a, b and c are schematic side-view illustrations of possible enteral connectors, or grips therefor, in accordance with the present invention.

In FIG. 1 of the drawings, a syringe 10 is of standard construction commonly employed in health services around the world. It has a standard connector outlet 12 which, at least in the UK health industry, will comprise a 6% luer taper. An integral latching mechanism, in the form of a control ring, that is frequently employed is not shown.

However, the syringe 10 has had permanently applied to its luer connector 12 a first converter 14′. The converter 14′ has a female luer inlet 16′ and is permanently fixed to the syringe 10 by any convenient means, for example, as by ultrasonic welding, adhesion or some other method.

The converter 14′ has a different male connector 18′ at its end remote from the inlet 16′. The “different” male end 18′ is merely different from the 6% luer connector 12 so that it could not mate with a 6% luer female connector. One possible form of different connector simply has a reduced diameter, but still comprises a 6% conically tapered fitting. With a sufficient reduction in diameter, it is clear that no connection is being effected when the different male is inserted into the standard female. Needless to say, the standard male cannot be fitted inside a reduced diameter different female. However, any alternative form of connection may be appropriate. For example, a standard 6% luer could be provided but with a latching mechanism in the form of a control element or ring 20′ which prevents connection to a standard female luer connector at least, not to one that does not have a connection feature that matches the control ring 20′. However, in the present case illustrated in FIG. 1, it is anticipated that the connector 18′ is different from the connector 12.

The control ring 20′ is axially slidable between a shoulder 22 and a rib 24 formed on the shaft of the converter 14′. The control ring 20′ has thread elements 26′.

A second converter 30 is also shown in FIG. 1. This comprises a female connector 32 adapted to fit the different male connector 18 of the first converter 14′. It has flange elements 34 adapted to fit with the thread elements 26′ of the control ring 20′. Thus, with the control ring 20′ slid leftwardly in the drawing, the female inlet 32 can be mated with the connector 18. To lock the arrangement, the thread elements 26′ can be engaged with the flange elements 34. The converter 30 is then releasably locked to the syringe 10.

The converter 30 at its other end comprises a standard male 6% luer connector 36 having an integral latching mechanism or control ring 38 provided with thread elements 40.

In this embodiment, the kit in accordance with this embodiment of the present invention further comprises another first converter 14. This differs from the first converter 14′ permanently connected to the syringe 10, only in its female luer inlet 16, which here is provided with flange elements 42 adapted to engage with thread elements 40 of a standard male 6% luer connector. Otherwise, the converter 14 is identical with the converter 14′ and has the same “different” male connector 18 and control ring 20. Indeed, the syringe 10 is usually provided with a control ring (not shown) corresponding to ring 38. In this event, if first converter 14 is identical to converter 14′, the latter is screwed onto to syringe 10 and permanent fixation thereon is easily effected by pouring adhesive into the control ring (38) around the 6% luer female end of the converter. This method ensures no adhesive can find its way into the bore of the converter or syringe, because a sealing connection isolating the bore of the arrangement is already made before the adhesive is introduced.

Finally, an hypodermic needle 50 is included in the kit shown and this comprises a needle 52 and a female connector 32′, corresponding with the female connector 32 of the second converter 30. It likewise has flange elements 34′ for engagement with thread elements 26 on control ring 20.

Thus the complete kit comprising syringe 10, second converter 30, first converter 14 and hypodermic needle 50 form the basis of a system intended for a particular area of medical applications. One such area is in respect of neuraxial investigations of human patients and for which special risks apply with regard to the injection of drugs in that region of the human body. It happens that there are a range of unique needle forms for various applications in this area and it is proposed that all such needles should now be provided with the different female connector 32′.

It is also proposed that converter 14, or at least the control ring 20 of the converter 14, that fits the needle 50, is brightly colour-coded. However, that alone is not sufficient, and the present invention provides both visual and tactile cues as to the nature of the application for which the system shown in FIGS. 2 to 4 is adapted. Both control rings 20 and 38 constitute grips by which the connection system of the present invention is operated. Both grips 20,38 are generally cylindrical and their outer surfaces are formed with four longitudinal spines 56, and encircling ribs 58, so that the whole grip 20,38 is suggestive of a human spine and rib cage. That is to say, the system is intended for neuraxial application.

What is more, ribs 58b, intermediate end ribs 58a, are of smaller diameter than end ribs 58a, so that the grip is suggestive of a pulling and pushing mechanism for effecting the connection between connectors. In this embodiment, the spines 56 are also mildly suggestive of a twisting motion, which is needed, of course, to complete locking between rings 20,38 and their male counterparts 42,34.

It is also anticipated that syringes 10 might be correspondingly coloured coded so that, henceforth, only such coded syringes and components would be employed in a neuraxial application.

Indeed, in time, syringe 10 will not be formed with a 6% luer connector 12, but rather will be provided ab initio with the different male connector 18 and control ring 20. Consequently, at that time the shape affordance is all that is required, although there is nothing to prevent colour coding also being employed, if desired.

The syringe 10 can be connected directly to the hypodermic needle 50. However, it is not always the case that a syringe is directly connected to the needle. Instead, intermediate components such as tubing or filters may be required. A standard filter 60 is shown in FIG. 1 and comprises a flat chamber 62 incorporating a filter disc 64. It has a standard 6% luer inlet 16′ and a standard 6% male outlet 36′.

To fit the filter 60 into the presently proposed scheme, a first converter 14 is screwed onto the male outlet 36′, while a second converter 30 is screwed onto the female 6% luer connector 16′ of the filter 60. The composite structure of the two converters 14, 30 and filter 60 in between, can then be interposed between the syringe 10 and the hypodermic needle 50. When adapted for connection into the neuraxial application, it carries the flag (by coded ring 20) demonstrating that it is for that application. Until the converters 14, 30 are so connected, however, components like the filter 60 cannot be interposed. A series of such components could be employed, each with a converter at either end. On the other hand, as long as a string of components joined together using their standard connectors have at each end the first and second converters 14,30 respectively, the string can be interposed between a syringe 10 and needle 50. However, it would be preferable to have, for example, at least a pair of first and second converters (joined together through their “different” connectors 18,32) interposed in the string so as to label the string as being the application for which that coding relates. Moreover, all the connections (other than those between “different” connectors) would be rendered undisconnectible, for example by gluing between a converter 14,30 and the component to which it is connected through its standard 6% luer connection. This will avoid the general risk of a neuraxial component being added to a standard tubing line.

FIG. 1 is a schematic illustration of some of the components of the present invention. However, FIGS. 2 and 3 illustrate preferred forms of first and second converters 14,30 respectively. The same reference numerals employed in FIG. 1 are employed in the converters shown in FIGS. 2 and 3.

In FIG. 4, a first converter in accordance with the present invention is permanently connected to a male connector 36′ of a standard component 52, only the connector of which is shown. The female connector 16, with its flange elements 42, is screwed into the threads 40 of the control ring 38 of the component 52 until a fluid tight seal is effected between the luer connection 16,36′. Then, adhesive 54 is poured between the inside of the control ring 38 and outside of female connector 16. When set, the adhesive permanently locks the flanges 42 in place and prevents disconnection. Alternatively, ultrasonic vibration may be applied between the connection 16,36′ to weld the components together.

While a first converter 14 is shown connected to a component 52 having a standard male connector 36, exactly the same joint, albeit 36,16′ (not shown), is made between a second converter 30 and a component having a standard female connector 16′.

FIGS. 2 to 4 show converters that are suitable for the application of the system of the present invention to neuraxial uses. The grips 20,38 associated with the connectors are suggestive of the human spine and rib cage and remind a user that only connections between similar components having similar grips will be successful. This affordance is provided both visually and tactually, and serves to prevent attempts at misconnection. FIGS. 5, 6 and 7 show other components of a neuraxial application of the present system having the same affordance. FIGS. 5a and b show an epidural needle hub 60. Wings 56′ of a grip 62 are large here to emphasise a twisting requirement to effect secure connection. Likewise, the ribs 58′ are clearly waisted (ie smaller in the centre) to emphasise also the pushing requirement to effect secure connection. FIGS. 7a, b and c show a neuraxial needle hub 64, which likewise has wings 56″ and ribs 58″. Four wings 56″are provided, as in the previous embodiments. In FIGS. 6a and b, a needle adaptor for neuraxial applications is shown, having the same wing and rib affordance. This is to adapt a standard needle to neuraxial applications.

However, to have value, such affordances need to be distinguishable from other applications and FIGS. 8a and b show potential connectors 70,72 for respiratory medical tubing. The connectors 70,72 comprise grips 74 which are bellows-like, suggestive of air flow. These are easily distinguishable from neuraxial applications, both visually and tactually. A button 76 also indicates a locking function, actuable by pressing the button.

FIGS. 9a, b and c, show connectors 78, 79 and 80 for use in enteral applications. These have grips 74,74′ and 74″ that are bulbous and are suggestive of the human colon. Grip 74′ has wings 56′″, to encourage twisting on connection. Grip 74″ is in the form of beads which tactually may be more distinguishable from the grips of the respiratory and neuraxial applications, while still being visually reminiscent of the human colon. Other applications can be envisaged having different grip designs.

The connectors of the present invention also comprise a stub, by means of which they are connected to the tubing or component of which they form a part. In the case of the converters 30,14 of FIGS. 1 to 4, the stub is the standard luer connection 36,38,40 and 16,42 respectively. However, in the case of needle hubs 60,64 of FIGS. 5 and 7, the stub is in the form of needle receptors 85 (needles not shown). Equally, where the connectors are formed directly on tubing or components, the stub will be that part of the connector that connects to the tubing or component, as the case may be.

Likewise, the connectors of the present invention include a key that uniquely permits one connector to connect to an appropriately keyed other connector. In the converters of FIGS. 1 to 4, the key is the reduced dimension luer connector. That is to say the key and male/female components of the connection are integral. However, there is no reason that this should necessarily be the case. Indeed, the system of the present invention could employ male/female interconnections that are standard luer fitments, the same for each application. In this event, there is a separate key that could, for example, comprise two spaced pins angularly disposed around a collar of one, say the female, component, and corresponding apertures on a collar of the male component. Then, if a female component from another application is given pins with a different angular disposition, this will prevent it connecting to the male component of the first application.

Within the scope of the present invention are components of the system that include a connector having a grip provided with application affordance. The system of the present invention includes all parts having a through bore for passage of fluid, including needles and containers for connection to the system.

No system is completely foolproof, and a determined person can get around all practical safeguards. However, given the impracticability of a foolproof system, one which provides obstacles that, to be overcome, requires unusual behaviour of the person concerned, is the next best thing. Such unusual behaviour (tampering with fittings to make them interconnect) tends to be noticed in a hospital environment and can cause alarms to be raised. In this respect, castellations 90 are provided on the ends of female connectors 30,64, as shown in FIGS. 3, 6 and 7. These ensure that leak paths 92, between the castellations 90, exist in the event that someone attempts to abut the end of a standard male luer fitting against the face of a female fitting according o the present invention, and effect a transfer of fluid. This added safety measure ensures that a miscreant will need to use adhesive tape to seal the gaps 92, or cut the castellations 90, in order to make a fluid transfer. Such an activity may be sufficient to create suspicion that an inappropriate procedure is being followed.

From the foregoing description, the present invention provides a simple route towards the adoption of the different connection system for the neuraxial, or indeed any other, application. From the beginning, it is anticipated that the needles 50 will be especially adapted for the different connector. Although it is proposed that the syringes 10 would be permanently adapted by the fixing of the first converter 14′ to the outlet 12, in due course it is anticipated that syringes would also be made having the different outlet 18. Finally, in time, components like the filter 60 will themselves be made with the different connectors 32,18 in place of the standard connectors 16′, 36′. However, not all of that needs to be implemented immediately. Instead, the system can be tried and tested and the economies of particular situations maximised before the system is integrated into all the components.

1. A system of medical small bore tubing for multiple different applications, the system in each application comprising connectors between tubing of the system or components of the system, wherein said connectors comprise:
a male component having a stub, a first key and a through-bore for the passage of fluid to be transported; and
a female component having a stub, a second key and a through-bore for the passage of fluid to be transported;
said male and female components being adapted to be interconnected in a fluid-tight manner with inter-engagement of said first and second keys, and said stubs being adapted for connection to tubing of the system or components of the system, and at least one of said male and female components having a grip; wherein, in each application:
a) the first and second keys are unique to each application of the system so that they prevent connection of a female component of one application to a male component of another application; and
b) said grip has application affordance unique to the application for which it is intended, the affordance comprising both visual and tactile cues; wherein misconnections between tubing and components of said different applications of the system are prevented and attempts by users to effect said misconnection are discouraged by said affordance of said grip.

2. A system as claimed in claim 1, wherein said application affordance comprises a shape of the grip that is suggestive of a part of a human body for which the application is intended.

3. A system as claimed in claim 2, wherein a first application is neuraxial, and said shape of the grip is generally cylindrical having a longitudinal spine and encircling ribs suggestive of the human spine and ribs.

4. A system as claimed in claim 2, wherein a second application is respiratory, and said shape of the grip is generally cylindrical having alternating frusto-conical sections suggestive of a bellows.

5. A system as claimed in claim 2, wherein a third application is enteral, and said shape of the grip is generally cylindrical with bulges down its length suggestive of the human colon.

6. A system as claimed in claim 2, wherein said visual and tactile cues of the application affordance are provided only by said shape of the grip.

7. A system as claimed in claim 1, wherein said grip also comprises a mechanism affordance unique to a method of interconnection between said male and female components.

8. A system as claimed in claim 7, wherein said method of interconnection comprises a twisting step; and wherein said mechanism affordance comprises a wing of said grip.

9. A system as claimed in claim 7, wherein said method of interconnection comprises a pushing step; and wherein said mechanism affordance comprises a waist of said grip.

10. A system as claimed in claim 7, wherein said method of interconnection comprises a locking step; and wherein said mechanism affordance comprises a button of said grip.

11. A kit of components of a medical small-bore tubing connection system as claimed in claim 1, the kit comprising:
a first converter having:
a through bore;
a standard female connector;
a different male connector element; and
a latching mechanism on the different male connector adapted to engage a flange of a corresponding female connector to which said different male connector is sealingly mateable; and
a second converter having:
a through bore;
a standard male connectors;
a different female connector that corresponds with the different male connector of said first converter; and
a flange adapted for engagement with the latching mechanism of said first converter.

12. A kit as claimed in claim 11, in which said standard connectors are 6% luer connectors.

13. A kit as claimed in claim 11, in which said different connectors are reduced-diameter 6% conical connectors.

14. A kit as claimed in claim 13, in which said reduced-diameter comprises about 3 mm for the end of the male connector, and about 3.3 mm for the opening of the female connector, and wherein each connector has a length of about 7.5 mm.

15. A kit as claimed in claim 11, further comprising a syringe, the syringe having a standard outlet; wherein the standard outlet is permanently secured to the first converter.

16. A kit as claimed in claim 15, wherein the standard outlet is permanently secured to the first converter by welding or adhering said first converter to said outlet.

17. A kit as claimed in claim 16, wherein said welding is ultrasonic welding.

18. A kit as claimed in claim 11, further comprising a hypodermic needle, said needle having said different female connector formed directly thereon.

19. A kit as claimed in claim 11, wherein said latching mechanism comprises a threaded collar and said flange comprises thread elements.

20. A kit as claimed in claim 19, wherein the latching mechanism on the first converter is axially slidable between limits, and is rotatably free.

21. A kit as claimed in claim 11, wherein the latching mechanism is visually coded to identify a class of medical applications for which it is intended.

22. A kit as claimed in claim 11, wherein the standard male connector of said second converter has an integral latching mechanism formed thereon adapted to co-operate with flange elements provided on the standard female connector of said first converter to lock said standard male and female connectors together.

23. A kit as claimed in claim 11, wherein said different female connector comprises a face having castellations; and wherein leak paths are provided between said castellations in the event that a standard male connector is butted against said face.

24. A syringe adapted for connection to the second converter of a kit as claimed in claim 11, the syringe comprising an outlet having a different male connector to a standard male connector and a latching mechanism on the different male connector adapted to engage a flange of a corresponding female connector to which said different male connector is sealingly mateable.

25. A component of medical tubing having a standard male connector and a standard female connector to which a first connector and a second connector of a kit as claimed in claim 11 have been connected.

26. A component as claimed in claim 25, wherein the standard female connector of said first converter comprises flange elements; and wherein the standard male connector of said second converter has an integral latching mechanism formed thereon adapted to co-operate with the flange elements on of the standard female connector of said first converter to lock said standard male and female connectors together, and wherein said connections have been rendered permanent by application of adhesive between a latching mechanism on the component and the standard female connector of the first converter and between the latching mechanism of the second converter and the female connector of the component.

27. A component as claimed in claim 25, which component is a filter, valve or tube junction.

28. A method of introducing into use a new connection system for an existing medical small bore tubing system that employs standard male and female connectors adapted to be sealingly mated together, said method comprising the steps of:
a) providing a plurality of kits as claimed in claim 11;
b) permanently connecting the standard female connectors of said first converters to the standard male connectors of components of said existing system; and
c) permanently connecting the standard male connectors of said second converters to the standard female connectors of components of said existing system.

29. A method as claimed in claim 28, wherein said permanent connection is by ultrasonic welding.

30. A method as claimed in claim 28, wherein the standard female connector of said first converter comprises flange elements; and wherein the standard male connector of said second converter has an integral latching mechanism formed thereon adapted to co-operate with the flange elements of the standard female connector of said first converter to lock said standard male and female connectors together; and wherein said permanent connection is by adhesion through adhesive disposed between the inside of said latching mechanism and the outside of said standard female connector.

31. An article of a medical small bore tubing system as claimed in claim 1, the article comprising: a connector having a male or female component, a stub, a grip, a key and a through-bore for the passage of fluid to be transported, said component being adapted to be connected in a fluid-tight manner with a corresponding component of another connector and with inter-engagement of said key with the key of said other component, and said stub being connected to said article; wherein said grip has application affordance unique to the application for which the article is intended, the affordance comprising both visual and tactile cues.
